(19) United States
(12) Patent Application Publication
(10) Pub. No.: US 2026/0305633 A1
(43) Pub. Date: Oct. 8, 2026

(54) NOVEL LACTOBACILLUS STRAINS

(71) Applicant: DSM IP ASSETS B.V., Maastricht (NL)

(72) Inventors: Paul KLAASSEN, Echt (NL); Jeroen SCHOEVERS, Echt (NL); Thomas Hendrik ECKHARDT, Echt (NL); Daniel Avraham SCHWARTZ, Echt (NL)

(21) Appl. No.: 19/480,543

(22) PCT Filed: May 6, 2024
(86) PCT No.: PCT/EP2024/062488

§ 371 Date: Oct. 31, 2025

(30) Foreign Application Priority Data
May 4, 2023 (EP) 23171715.8

(51) Int. Cl. A23C 9/123; A23C 13/16; A23C 19/06; C12N 1/20; C12R 1/225

(52) U.S. Cl. CPC A23C 9/1234; A23C 13/16; A23C 19/062; C12N 1/20; C12R 2001/225

(57) ABSTRACT

The present invention relates to novel Lactobacillus strains with extended shelf-life properties, i.e., capability to extend the shelf-life at ambient temperature of a food product, such as e.g., a fermented milk product, when added to such products, without heavily diminishing the antimicrobial, antifungal and/or anti-yeast effect of said strains. The invention furthermore relates to a method of generating such novel strains, particularly generation via adaptive evolution.

The present invention relates to novel Lactobacillus strains with extended shelf-life properties, i.e., capability to extend the shelf-life at ambient temperature of a food product, such as e.g., a fermented milk product, when added to such products, without heavily diminishing the antimicrobial, antifungal and/or anti-yeast effect of said strains. The invention furthermore relates to a method of generating such novel strains, particularly generation via adaptive evolution.

Lactic acid bacteria (LAB) are known for their role in the preparation of fermented foods, such as for instance cheese, yoghurt, meat, and vegetable-based products.

Fermented foods, such as fermented food products, can be susceptible to the growth of undesired microorganisms, such as yeast(s), mould(s) and (pathogenic) bacteria. Fungi, such as moulds and yeasts, could grow abundantly in fermented food products, even under cold conditions.

Food grade chemical preservatives such as potassium sorbate and benzoate are traditionally adequate measures to prevent the undesired growth of yeasts, moulds, and bacteria. However, a drawback of these food grade chemical preservatives is that they are non-natural products and thus the products preserved with such a food grade chemical preservative do not have a clean label. This is undesirable in view of the increasing demand for natural products.

As an alternative, LAB with protective characteristics can be used. Fermented milk products often comprise LAB. During fermentation, lactic acid and other organic compounds are being produced by the LAB, thereby reducing the pH of the food product, and consequently making it unfavorable to the growth of undesired microorganisms, such as yeast(s), mould(s) and (pathogenic) bacteria. The use of LAB strains to prevent said undesired growth of contaminants, such as e.g., yeast, mould or pathogenic bacteria, is also known as bioprotection.

The bioprotective effect of LAB strains contributing to the production of a milk-derived bioactive peptide, which shows growth-inhibitory activity against Debaromyces hansenii, has been described by McNair et al. (FEMS Yeast Research, 2018, Vol. 18, No. 8).

In EP3279312 a single Lactobacillus rhamnosus species CBS 141584 is described having antimicrobial, antifungal and/or anti-yeast properties.

The effect on bioprotection using a combination of strains from Lactobacillus rhamnosus and Lactobacillus paracasei has been shown in the past, see e.g. WO2013/153074 or WO2012/136830.

An example of commercial use of a combination of Lactobacillus rhamnosus and Lactobacillus paracasei is FreshQ®, which is a bioprotective culture for fermented milk products available from Chr. Hansen, Denmark.

Although the presence of LAB in fermented milk products is desired, particularly with regards to extension of shelf-life and enhanced safety of food, more particularly in view of an increasing demand in the market for products that are suitable for transport, distribution and/or storage at ambient temperatures, it is a challenge to use them to reduce the undesired, non-controlled growth of contaminant yeasts, moulds and bacteria in fermented milk products.

The use of bioprotective cultures consisting of combinations of different species may increase the risk that the additional species introduce flavor to a food product, such as a fermented milk product. This is a disadvantage because it limits the applications of the bioprotective culture. Further developments were made to prepare LAB strains with improved characteristics on this point.

A further well-known problem in the production of fermented milk products is post-acidification. Post-acidification or post-fermentation acidification is an undesired process in fermented milk products whereby continued acidification beyond the optimal range occurs during shelf-life of the fermented milk product. The post-acidification predominantly takes place during shelf-life of the fermented milk product. It may for example take 5 to 12 days before a customer opens a package. If during this period the product acidifies further this can affect the perceived taste in a detrimental manner, even if the change is pH is small.

Thus, there is still a need in dairy industry to improve bioprotection and shelf-life of products, particularly fermented milk products, more particularly extension of the shelf-life of such products at ambient temperature.

Surprisingly, we now found a method to extend the shelf-life of food products, particularly dairy products such as e.g., fermented milk products, resulting in bioprotective LAB strains showing improved post-acidification profile especially at ambient temperature.

Thus, the present invention is directed to a process for obtaining mutant LAB strains, preferably Lactobacillus bacterial strains, said mutants being capable of reducing post-acidification in a fermented milk product, such as e.g., yogurt, in comparison with a parent or mother Lactobacillus bacterial strain, particularly a bioprotective strain, such as e.g., a strain selected from Lactobacillus casei, Lactobacillus rhamnosus, Lactobacillus plantarum or Lactobacillus paracasei, particularly a mutant strain derived from strain L. casei CBS 148322 as parent strain, more particularly a mutant strain deposited as CBS 151609, said method comprising subjecting the parent LAB strain to an evolution process, including but not limited to a method wherein the parent or ancestral strain is subjected to gradual or abrupt increase of temperature, particularly to a gradual increase in temperature, also known as adaptive laboratory evolution (ALE), said adaptive evolution process comprising a series of evolutionary changes, wherein each next descendent strain of the preceding strain is subjected to a higher environmental temperature than the preceding strain.

As used herein, the terms “mother strain”, “ancestral strain”, “ancestor strain” or “parent strain” are used interchangeably herein. The Lactobacillus bacterial strain to be used as mother strain might be selected from a Lactobacillus rhamnosus strain, a Lactobacillus plantarum strain, a Lactobacillus paracasei strain or a Lactobacillus casei strain, most preferably a strain of Lactobacillus casei. A particularly useful ancestral strain subject to mutations as described herein is selected from a Lactobacillus, preferably Lactobacillus casei, more preferably L. casei CBS 148322 as described in PCT patent application no. PCT/EP2022/080223.

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or more than one (i.e. to one or at least one) of the grammatical object of the article. By way of example, “an element” may mean one element or more than one element. When referring to a noun (e.g., a compound, an additive, etc.) in the singular, the plural is meant to be included. Thus, when referring to a specific moiety, e.g., a “strain”, this means “at least one” of that strain, e.g., “at least one strain”, unless specified otherwise.

When referring to a compound of which several isomers exist (e.g., a D and an L enantiomer), the compound in principle includes all enantiomers, diastereomers and cis/trans isomers of that compound that may be used in the particular aspect of the invention; in particular when referring to such as compound, it includes the natural isomer(s).

As used herein, the term “lactic acid bacteria” (LAB) or “lactic bacteria” refers to food-grade bacteria producing lactic acid as the major metabolic end-product of carbohydrate fermentation. These bacteria are related by their common metabolic and physiological characteristics and are usually Gram positive, low-GC, acid tolerant, non-sporulating, non-respiring, rod-shaped bacilli, or cocci. During the fermentation stage, the consumption of sugars by these bacteria causes the formation of lactic acid and reduces the pH. These bacteria are thus responsible for the acidification and in some cases (e.g., in case of milk fermentations) for the texture of the fermented product. As used herein, the term “LAB” or “lactic bacteria” encompasses, but is not limited to, bacteria belonging to the genus of Lactobacillus spp., Bifidobacterium spp., Streptococcus spp., Lactococcus spp., such as Lactobacillus delbruekii subsp. bulgaricus, Streptococcus thermophilus, Lactobacillus lactis, Bifidobacterium animalis, Lactococcus lactis, Lactobacillus casei, Lactobacillus plantarum, Lactobacillus paracasei, Lactobacillus rhamnosus, Lactobacillus helveticus, Lactobacillus acidophilus and Bifidobacterium breve.

The term “milk” is intended to encompass milks from mammals, from plant sources or recombinant produced milk. Preferably, the milk is from a mammal source. Mammal sources of milk include, but are not limited to cow, sheep, goat, buffalo, camel, llama, mare and deer. In a preferred embodiment, the milk is from a mammal selected from the group consisting of cow, sheep, goat, buffalo, camel, llama, mare and deer, and combinations thereof. Plant sources of milk include, but are not limited to, milk extracted from soybean, pea, peanut, barley, rice, oat, quinoa, almond, cashew, coconut, hazelnut, hemp, sesame seed, sunflower seed, and/or mixtures thereof. In addition, the term “milk” refers to not only whole milk, but also skim milk or any liquid component derived thereof.

As used in the present specification, the term “fermented milk product” refers to a product that has been fermented with LAB. Examples of LAB are Streptococcus thermophilus and Lactobacillus delbruekii subsp. bulgaricus, but also, optionally, other microorganisms such as for instance Lactobacillus delbruekii subsp. lactis, Bifidobacterium animalis subsp. lactis, Lactococcus lactis, Lactobacillus acidophilus and Lactobacillus casei, or any microorganism derived therefrom. The fermentation process increases the shelf-life of the product while enhancing and improving the digestibility of milk. Many different types of fermented milk products can be found in the world today. Examples are soured milk (e.g., buttermilk), sour cream and yogurt.

The term “starter culture” (also referred to as “starter”) as used herein refers to a composition comprising one or more LAB strains, which are responsible for the acidification of the milk or milk base. Starter cultures compositions may be fresh (liquid), frozen or freeze-dried. Freeze dried cultures need to be regenerated before use. For the production of a fermented milk product, the starter cultures composition is usually added in an amount from 0.01 to 3%, preferably from 0.01 and 0.02% by weight of the total amount of milk or milk base.

The term “mutant” should be understood as a strain derived from a strain of the invention, also called mother strain or ancestral strain, obtained by means of e.g., chemical mutagenesis, radiation, or genetic engineering. Mutants may even arise in a population that is not actively treated to obtain mutants, by means of errors during DNA replication (so-called “spontaneous” mutations) and are also included herein. Many methods are known in the art for obtaining mutants and methods for selecting mutants with desired properties are well known. A particularly useful method for generation of mutant strains in the scope of the present invention is the adaptive laboratory evolution (ALE) technique further described herein. Preferably, the mutant is functionally equivalent to the original or mother strain, in the sense that the mutant has the same or preferably improved properties, such as e.g., the ability to produce antifungal or antimicrobial compounds. Preferably, any mutant as described herein has a nucleic acid sequence with at least about 70% identity, more preferably with at least about 80, 90, 95, 99 or 99.9% identity with the nucleic acid sequence of the ancestral strain from which it is derived. Preferably, any mutant as described herein has an improved post-acidification property at ambient temperature as compared to the parent strain.

The term “thermophile” or “thermophilic” herein refers to LAB that thrive well at temperatures above about 41° C. The most useful thermophilic lactic acid bacteria include Lactobacillus spp, and Streptococcus ssp. Hence, a “thermophilic fermentation” herein refers to a fermentation that is being executed at a temperature above about 35° C., for example between about 35° C. and about 45° C., such as e.g., at about 42° C.

The term “mesophile” or “mesophilic” herein refers to LAB that thrive best at temperatures lower than about 41° C., such as e.g., between about 15° C. and about 40° C. Examples of mesophilic lactic acid bacteria with industrial relevance include for instance Lactococcus ssp, and Leuconostoc ssp. Hence, a “mesophilic fermentation” herein refers to a fermentation that is being done at a temperature between about 20° C. and 36° C., such as e.g., at about 28° C.

The term “fermentation” herein refers to a metabolic process wherein sugar(s) are being converted into acids, gases, or alcohol. Fermentation occurs in many different cell types, such as e.g., yeasts and bacteria. Preferably, fermentation comprises the conversion of lactose into lactic acid.

“Undesired microorganisms”, “undesired contaminants” and “contaminants” herein refer to the occurrence of microorganisms, such as bacteria, yeasts, moulds, or a combination thereof, which bring about a negative perception of the food. The contaminant may be pathogenic, may have the ability to deteriorate food products or, give rise to an unpleasant smell, taste, or appearance of the food product. The strain of the invention is providing a solution in the prevention of the appearance and/or growth of such contaminants by inhibiting and/or preventing their growth upon entry in the fermented milk matrix, such as e.g., a yogurt or a sour cream product. The prevention of the growth of contaminants due to the action or the presence of Lactobacillus bacterial strain can be expressed by e.g., a lower number of contaminant cell counts in a fermented milk product prepared with the present Lactobacillus bacterial strain, compared to a similar product, without the presence of said Lactobacillus bacterial strain.

As used herein, the term antimicrobial, an antifungal or an anti-yeast composition means a composition suitable for providing an antimicrobial, an antifungal or an anti-yeast efficacy.

The terms “bioprotective” or “protective” strains in relation to LAB strains are used interchangeably herein and refer to the properties of said strains to control, particularly inhibit or delay, the growth of undesired microorganism such as e.g., yeast, moulds and other (pathogenic) bacteria. Said process is also referred to as “bioprotection” or “biopreservation”.

According to one aspect of the present invention, the mutant Lactobacillus bacterial strains as described herein are generated from a parent strain by means of Adaptive Laboratory Evolution (ALE). ALE can be described as a laboratory method in which microbial cells are harnessing biology to self-optimize by natural (or induced) mutations through selection. Adaptation of a microbial culture to a new environment necessarily involves the enhancement of certain traits, leading to improved function and an increase in fitness under the applied conditions. Detailed guidance on experimental evolution of microbes can be found in Bachmann et al. (FEMS Microbiology Reviews, 2017, Vol. 41, No. Supp 1) using Escherichia coli, concluding that, while lots of progress on genome dynamics and cellular resource allocation may be made in recent years, there are still many open questions on how different constraints during the evolution influence such trade-offs and eventually the fitness of an organism in a particular environment.

The present invention conveniently provides a method for obtaining a mutant LAB, more preferably a Lactobacillus bacterial strain, preferably capable of reducing post-acidification in a fermented milk product in comparison to the respective ancestral Lactobacillus bacterial strain, wherein the method comprises subjecting the parent LAB, respectively Lactobacillus bacterial strain, to ALE, said ALE comprising a series of evolutionary changes wherein each next descendent strain of a preceding strain is subjected to, and preferably grown at, a higher environmental temperature than the preceding strain. Different forms of ALE exist and have been described (see e.g., Dragosits and Mattanovich, 2013, Microb Cell Fact 12, 64. [https://doi.org/10.1186/1475-2859-12-64]; Sandberg et al., 2019, Metabolic Engineering 56, 1-16. [https://www.sciencedirect.com/science/article/pii/S1096717619301533]; WO2019/043055; Bennett and Lenski, 2007, PNAS, vol. 104, suppl. 1, 8649-8654. [www.pnas.orgcgidoi10.1073pnas.0702117104]).

More preferably, the invention provides a method for obtaining a mutant LAB strain, preferably capable of reducing post-acidification in a fermented milk product in comparison to the respective ancestral LAB strain, wherein the method comprises subjecting an LAB strain to ALE, particularly wherein the LAB strain has protective properties, more preferably wherein the LAB strain is selected from a Lactobacillus strain, even more preferably wherein the LAB strain is selected from strain CBS 148322, said ALE comprising a series of evolutionary changes wherein each next generation descendent of a preceding parent strain is subjected to, and preferably grown at, an environmental or incubation temperature that is at least 1° C. higher than the environmental or incubation temperature at which the preceding parent strain was grown, wherein the LAB strain preferably is a Lactobacillus bacterial strain. ALE consists of several rounds of mutations or adaptions as described herein, preferably with 1 to 10 rounds, such as e.g., 1 to 5, 1 to 7, 1 to 8 rounds, with characterization of the obtained mutants after the final round.

Thus, the present invention includes a Lactobacillus bacterial strain as described herein, particularly a mutant strain derived from strain CBS 148322, said strain being obtained, obtainable or produced by the above method using ALE.

A particularly preferred mutant Lactobacillus bacterial strain has been deposited as CBS 151609 on 2 May 2024 at the Westerdijk Fungal Biodiversity Institute (CBS, formally named the Centraalbureau voor Schimmelcultures), Uppsalalaan 8, 3508 AD Utrecht, The Netherlands under the provisions of the Budapest Treaty.

The present invention furthermore refers to Lactobacillus bacterial strain deposited as CBS 149825 on 8 Mar. 2023 at the Westerdijk Fungal Biodiversity Institute (CBS, formally named the Centraalbureau voor Schimmelcultures), Uppsalalaan 8, 3508 AD Utrecht, The Netherlands under the provisions of the Budapest Treaty.

It was surprisingly found that said Lactobacillus bacterial strain CBS 151609 provides for an improved post-acidification, especially at ambient temperature (20° C.), compared to the ancestral strain CBS 148322 and also as compared to further mutant strains derived from several rounds of ALE (see Examples).

Preferably, the Lactobacillus bacterial strain obtainable by ALE as described herein increases the pH of a fermented milk product, comprising said Lactobacillus bacterial strain, during storage after fermentation in comparison to a fermented milk product, comprising a Lactobacillus bacterial strain added as bioprotective agent, particularly in comparison to an ancestral strain deposited as CBS 148322, wherein the increase in pH is at least by a value of 0.1, such as e.g. in the range of 0.1 to 0.2 or more when using the mutant strain, such as e.g. strain CBS 151609, and wherein the increase in pH is determined after storage of the fermented milk product over about 13-14 days at 20° C., fermented with a starter culture. Particularly said starter culture is preferably comprising or consisting of Streptococcus thermophilus and Lactobacillus delbruekii subsp. bulgaricus, particularly with an inoculation of the fermented milk product with the starter culture of about 1 U/1000 L. Particularly, the bioprotective Lactobacillus strain, such as e.g., strain CBS 148322 or preferably strain CBS 151609, are added to the fermented milk product in a concentration of at least about 10⁷ CFU/g.

The Lactobacillus bacterial strains according to the present invention such as obtainable from strain CBS 148322 and as described herein, particularly the mutants as described herein, more particularly mutant strain CBS 151609, preferably have antimicrobial, antifungal and/or anti-yeast effect, wherein through the application of ALE the shelf-life with regards to post-acidification is improved, particularly if said mutant strain is added as bioprotective to a dairy product such as e.g. a yogurt.

Preferably, in 12 wt % reconstituted skim milk (RSM) and at an inoculation rate of 10⁷ CFU/g, when combined with a starter culture comprising or consisting of Streptococcus thermophilus and Lactobacillus delbruekii subsp. bulgaricus, the mutant Lactobacillus bacterial strain obtainable via ALE as described herein, preferably strain CBS 151609, added as protective adjunct has an acidification profile exhibiting a Time To Reach (“TTR”) pH 4.6 in the range of 400 to 350 minutes or less.

Preferably, the Lactobacillus bacterial strains according to the present invention, particularly the mutant strains obtainable via ALE, more particularly strain CBS 151609, and as described herein are suitable for providing an antifungal effect and/or an anti-yeast effect, more preferably without introducing a flavor effect.

Particularly, the novel Lactobacillus bacterial strain(s) as obtainable by ALE as described herein is a mutant strain derived from the ancestral strain CBS 148322 and particularly includes a strain deposited as strain CBS 151609, said novel strains having improved post-acidification properties as well as antimicrobial, antifungal and/or anti yeast properties comparable to the ancestor strain, such as strain CBS 148322.

Mutants as used in the above context mean Lactobacillus bacterial strains which are derived/obtained from CBS 148322, having mutation(s) in comparison with Lactobacillus bacterial strain CBS 148322, wherein the mutation(s) do not alter the bioprotective phenotype of the derived Lactobacillus bacterial strain. Preferably, the mutant strain has the same or improved antimicrobial antifungal and/or anti-yeast properties as the mother strain CBS 148322. Preferably, the derived Lactobacillus bacterial strain is suitable for providing an antifungal effect and/or an anti-bacterial effect, as found for or like strain CBS 148322 or even better. The mutant derived from CBS 148322 might have at least about 80%, such as e.g., about 90, 95, 100% or more of the antimicrobial, antifungal, and/or anti-yeast effect compared with strain CBS 148322, if compared under equal conditions. In a particularly preferred embodiment, the present bioprotective Lactobacillus bacterial strain is a strain selected from Lactobacillus strain CBS 148322, further most preferably the mutant strain with improved post-acidification properties as described herein is selected from strain CBS 151609 but furthermore includes other mutant strains derived from strain CBS 148322 as described herein.

In one embodiment, the mutant Lactobacillus bacterial strains as defined herein and obtainable via ALE, particularly strain CBS 151609, has bioprotective properties, such as e.g., being capable of limiting the fungal growth of known contaminants in dairy industry, including but not limited to strains of Aspergillus, Fusarium, Penicillium, Toluraspora, Debaryomyces, Pichia, Zagosaccharomyces, Yarrowia, Kluyveromyces, Saccharomyces or other known moulds and yeast.

Preferably, the Lactobacillus bacterial strains according to this invention, such as Lactobacillus bacterial strain CBS 148322 and mutants thereof, more preferably mutant strain CBS 151609, are produced, supplied or otherwise present in frozen, dried, or freeze-dried form.

Preferably, the Lactobacillus bacterial strains according to the present invention and as described herein, such as Lactobacillus bacterial strain CBS 148322 and mutants thereof, more preferably mutant strain CBS 151609, are dosed, each individually, in an amount of 10⁶ CFU/g to 10⁸ CFU/g of a medium or substrate, such as for example milk.

In one aspect, this invention provides an antimicrobial and/or antifungal and/or anti-yeast composition comprising a Lactobacillus bacterial strain as described herein, such as e.g., a composition comprising Lactobacillus bacterial strain CBS 148322 and/or mutants thereof, preferably the mutant strain CBS 151609, comprised in such composition, particularly a composition comprising a mutant Lactobacillus strain selected from a strain deposited as CBS 151609, wherein preferably said mutant strain has the same or improved antimicrobial, antifungal and/or anti-yeast properties as the strain originating from, such as ancestral strain CBS 148322.

Preferably, the amount of the present Lactobacillus bacterial strain as described herein, such as e.g., Lactobacillus bacterial strain CBS 148322 and/or mutants thereof, preferably the mutant strain CBS 151609, in the antimicrobial, antifungal and/or anti-yeast composition is sufficient to provide an antimicrobial, an antifungal or an anti-yeast effect. This enables the antimicrobial, antifungal or anti-yeast composition suitable to provide an antimicrobial, an antifungal or an anti-yeast effect.

More preferably, the antimicrobial and/or antifungal and/or anti-yeast composition comprises both (i) a mutant Lactobacillus bacterial strain as described above, particularly strain CBS 151609, and (ii) a starter culture, such as one or more other lactic acid strains, such as for example one or more Streptococcus thermophilus strains and/or one or more Lactobacillus delbruekii subsp. bulgaricus strains. Optionally, additional, preferably bioprotective, strains selected from Lactobacillus casei strain, Lactobacillus paracasei strain, Lactobacillus plantarum strain and/or a Lactobacillus rhamnosus strain, might be part of said composition.

In a preferred embodiment, the present antimicrobial, antifungal and/or anti-yeast composition comprises:
(i) a mutant Lactobacillus bacterial strain, particularly deposited as CBS 151609, derived from the ancestor strain CBS 148322 wherein preferably said mutant has the same or improved antimicrobial, antifungal and/or anti-yeast properties as the mother strain, particularly as strain CBS 148322; and
(ii) optionally additional, preferably bioprotective, Lactobacillus casei strain, Lactobacillus paracasei strain, Lactobacillus plantarum strain and/or a Lactobacillus rhamnosus strain; and
(iii) optionally one or more other lactic acid strains, such as one or more Streptococcus thermophilus strains and/or one or more Lactobacillus delbruekii subsp. bulgaricus strains.

Preferably, the amount of the Lactobacillus bacterial strain(s) according to the present invention and as described herein, such as e.g., Lactobacillus bacterial strain CBS 148322 and/or mutants thereof, more preferably the mutant strain CBS 151609, comprised in the antimicrobial, antifungal and/or anti-yeast composition is sufficient to provide an antimicrobial, an antifungal or an anti-yeast effect. This enables the antimicrobial, antifungal or anti-yeast composition suitable to provide an antimicrobial, an antifungal or an anti-yeast effect.

The present antimicrobial, antifungal and/or anti-yeast composition preferably comprises the Lactobacillus bacterial strain(s) as described herein, such as e.g., a composition comprising Lactobacillus bacterial strain CBS 148322 and/or mutants thereof, preferably the mutant strain CBS 151609, and according to all aspects of the present invention in a concentrated form including frozen, dried or freeze-dried concentrates.

The present antimicrobial, antifungal or anti-yeast composition may further comprise components such as cryoprotectants and/or conventional additives including nutrients such as yeast extracts, sugars, and vitamins, e.g., vitamin A, C, D, K, or vitamins of the vitamin B family. Suitable cryoprotectants that may be added to the composition of the invention are components that improve the cold tolerance of the microorganisms, such as mannitol, sorbitol, sodium tripolyphosphate, xylitol, glycerol, raffinose, maltodextrin, erythritol, threitol, trehalose, glucose, sucrose, and/or fructose. Other additives may include, e.g., carbohydrates, flavors, minerals, salts, and/or enzymes (e.g., rennet, lactase and/or (phospho) lipase).

More preferably, the present antimicrobial, antifungal or anti-yeast composition is packed. Preferably, in a package which is suitable for shipment and/or storage of the present antimicrobial, antifungal or anti-yeast composition for at least about 1 month, such as at least about 3 months. Preferably, the amount of antimicrobial, antifungal or anti-yeast composition in the package is at least about 50, 100 or 500 grams, such as, e.g., at least about 50 to 500 grams, such as e.g., at least about 100 to 500 grams.

In a further aspect, the present invention relates to a food product comprising the Lactobacillus bacterial strain(s) as described herein, such as e.g., a food product comprising Lactobacillus bacterial strain CBS 148322 and/or mutants thereof, preferably the mutant strain CBS 151609, and according to all aspects of the present invention optionally other LAB strains, or the present antimicrobial and/or antifungal composition.

The present invention relates to a food product comprising an amount of the Lactobacillus bacterial strain(s) according to the present invention and as described herein, such as e.g., a food product comprising Lactobacillus bacterial strain CBS 148322 and/or mutants thereof, preferably the mutant strain CBS 151609, which is effective for imparting antimicrobial properties to the food product. More preferably, wherein the presence of the Lactobacillus bacterial strain(s) as described herein does not introduce a flavor to the food product.

Preferably, the present food product has a flavor profile which is comparable or indistinguishable from the food product, or from the same food product, which does not comprise the present Lactobacillus bacterial strain, particularly which does not comprise mutant strains as described herein, such as e.g., the preferred strain CBS 151609. In other words, the presence of the novel Lactobacillus bacterial strain and optionally any further additional strain does not introduce a flavor to a food product to which the Lactobacillus bacterial strain and optionally the further strain is added.

The mutant Lactobacillus bacterial strains according to the present invention, preferably the mutant strain CBS 151609, might be used for providing an antimicrobial and/or antifungal and/or anti-yeast effect in a food product, preferably in a fermented milk product.

Preferably, the present food product as described herein comprising the Lactobacillus bacterial strains according to the present invention and as described herein, such as e.g., a food product comprising Lactobacillus bacterial strain CBS 148322 and/or mutants thereof, preferably the mutant strain CBS 151609, is a fermented milk product. More preferably a mesophilic or thermophilic fermented milk product. Most preferably, the fermented milk product is a yogurt. Examples of a fermented milk product include regular yoghurt, plain yogurt, low fat yoghurt, nonfat yoghurt, tvarog, kefir, dahi, ymer, buttermilk, butter, sour cream, and sour whipped cream. Another preferred example of a fermented milk product is cheese. For example, fresh cheeses, un-ripened cheeses, or curd cheeses. Alternatively, the fermented milk product is a ripened cheese. The food product can also be a milk, whey, milk powder or whey powder.

According to yet another aspect, the present invention relates to a method for manufacturing a food product comprising adding at least one Lactobacillus bacterial strain as described herein, preferably adding mutant strains, more preferably at least the mutant strain CBS 151609, or the present antimicrobial composition, antifungal composition, or anti-bacterial composition, during manufacture of the food product. Preferably, the Lactobacillus bacterial strain according to the present invention and as described herein, preferably mutant strains, more preferably at least the mutant strain CBS 151609, is added to the milk together with LAB used for fermentation of the milk. Even more preferably, the present method comprises a step of fermenting milk with LAB. The present inventors found that a high efficacy against yeast and moulds can be obtained if the Lactobacillus bacterial strain as described herein, such as e.g., Lactobacillus bacterial strain CBS 148322 and/or mutants thereof, preferably the mutant strain CBS 151609, and according to the present invention is present during fermentation of the food product.

Thus, in a preferred embodiment, the method comprises one or more fermentation steps. Preferably, the method comprises fermenting a milk substrate with a starter culture comprising at least one strain of the genera selected from Lactobacillus, Streptococcus, Lactococcus and/or Leuconostoc. The present step of fermenting a milk substrate can be fermenting under mesophilic or under thermophilic conditions. More preferably, the present method is a method for manufacturing yogurt comprising adding at least one Lactobacillus bacterial strain according to the present invention, such one mutant strain as described herein, preferably at least the mutant strain CBS 151609, or the present antimicrobial composition, antifungal composition, or anti-bacterial composition, during fermentation of milk with a starter culture comprising Streptococcus thermophilus and Lactobacillus delbrueckii subsp. bulgaricus. Alternatively, the present method is a method for manufacturing sour cream comprising adding at least one Lactobacillus bacterial strain according to the present invention, or the present antimicrobial composition, antifungal composition, or anti-bacterial composition, during fermentation of milk with a starter culture comprising Lactococcus lactis subsp. cremoris and Lactococcus lactis subsp. lactis.

According to another aspect, the present invention relates to the use of the inventive Lactobacillus bacterial strain as described herein, such as e.g., Lactobacillus bacterial strain CBS 148322 and/or mutants thereof, preferably the mutant strain CBS 151609, for providing an antimicrobial effect in food products, preferably fermented milk products, preferably for providing an antifungal effect and/or an anti-yeast effect in food products, preferably fermented milk products. More preferably wherein the food product is a fermented milk product. More preferably a fermented milk product such as various types of regular yoghurt, low fat yoghurt, nonfat yoghurt, kefir, dahi, ymer, buttermilk, butter, sour cream, and sour whipped cream. Other examples of a fermented milk product, wherein such Lactobacillus bacterial strain as described herein can be used is cheese. For example, fresh cheeses, un-ripened cheeses, or curd cheeses. Alternatively, the fermented milk product is a ripened cheese.

The herein described antimicrobial, antifungal and/or anti-yeast composition preferably comprises the Lactobacillus bacterial strain(s) according to all aspects of the invention and as described herein in a concentration of viable cells, which is, each individually, preferably in the range of 10⁴ to 10¹³ cfu (colony forming units) per gram of the composition including at least about 10⁴ cfu per gram of the composition, such as at least about 10⁵ cfu/g, e.g., at least about 10⁶ cfu/g, such as at least about 10⁷ cfu/g, e.g., at least about 10⁸ cfu/g, such as at least about 10⁹ cfu/g, e.g., at least about 10¹⁰ cfu/g, such as at least about 10¹¹ cfu/g.

Thus, the antimicrobial, antifungal or anti-yeast composition of the invention is preferably present in a frozen, dried, or freeze-dried form, e.g., as a Direct Vat Culture (DVC). However, as used herein the composition may also be a liquid that is obtained after suspension of the frozen, dried, or freeze-dried cell concentrates in a liquid medium such as water, milk, or PBS buffer. Where the composition of the invention is a suspension, the concentration of viable cells is in the range of 10⁴ to 10¹² cfu (colony forming units) per ml of the composition including at least about 10⁴ cfu per gram of the composition, such as at least about 10⁵ cfu/ml, e.g., at least about 10⁶ cfu/ml, such as at least about 10⁷ cfu/ml, e.g., at least about 10⁸ cfu/ml, such as at least about 10⁹ cfu/ml, e.g., at least about 10¹⁰ cfu/ml, such as at least about 10¹¹ cfu/ml.

In a further preferred embodiment, the herein described food product comprises the Lactobacillus bacterial strain(s) according to the present invention and as described herein, such as e.g., a food product comprising Lactobacillus bacterial strain CBS 148322 and/or mutants thereof, preferably the mutant strain CBS 151609, in an amount, each individually, which is in the range of 10⁴ to 10¹² cfu (colony forming units) per gram of the food product including at least about 10⁴ cfu per gram of the food product, such as at least about 10⁵ cfu/g, e.g., at least about 10⁶ cfu/g, such as at least about 10⁷ cfu/g, e.g., at least about 10⁸ cfu/g, such as at least about 10⁹ cfu/g, e.g., at least about 10¹⁰ cfu/g, such as at least about 10¹¹ cfu/g of the food product. More preferably, such food product comprises the Lactobacillus bacterial strain(s) according to the present invention and as described herein in an amount, each individually, which is in the range of 10⁴ to 10¹² cfu (colony forming units) per cm² surface of the food product including at least about 10⁴ cfu/cm² of the food product, such as at least about 10⁵ cfu/cm², e.g., at least about 10⁵ cfu/cm², such as at least about 10⁷ cfu/cm², e.g., at least about 10⁸ cfu/cm², such as at least about 10⁹ cfu/cm², e.g., at least about 10¹⁰ cfu/cm², such as at least about 10¹¹ cfu/cm² surface of the food product.

In a further preferred embodiment, the herein described food product optionally comprises further bioprotective strains such as, e.g., Lactobacillus casei strain, Lactobacillus paracasei strain, Lactobacillus plantarum strain and/or Lactobacillus rhamnosus strain, and, if present, each individually, in an amount which is in the range of 10⁴ to 10¹² cfu (colony forming units) per gram of the food product including at least about 10⁴ cfu per gram of the food product, such as at least about 10⁵ cfu/g, e.g., at least about 10⁶ cfu/g, such as at least about 10⁷ cfu/g, e.g., at least about 10⁸ cfu/g, such as at least about 10⁹ cfu/g, e.g., at least about 10¹⁰ cfu/g, such as at least about 10¹¹ cfu/g of the food product. More preferably, such food product comprises the further or additional Lactobacillus strain in an amount which is in the range of 10⁴ to 10¹² cfu (colony forming units) per cm² surface of the food product including at least about 10⁴ cfu per cm² of the food product, such as at least about 10⁵ cfu/cm², e.g., at least about 10⁵ cfu/cm², such as at least about 10⁷ cfu/cm², e.g., at least about 10⁸ cfu/cm², such as at least about 10⁹ cfu/cm², e.g., at least about 10¹⁰ cfu/cm², such as at least about 10¹¹ cfu/cm² surface of the food product.

In a preferred embodiment, the present invention relates to the use of the inventive Lactobacillus bacterial strain as described herein, such as e.g., Lactobacillus bacterial strain CBS 148322 and/or mutants thereof, preferably the mutant strain CBS 151609, for providing an antimicrobial effect in silage, preferably for providing an antifungal effect and/or an anti-yeast effect in silage.

As explained below and illustrated by the examples, the invention advantageously allows for a bioprotective solution which not only affords a favorable shelf-life stability but also has reduced post-acidification compared to prior art compositions. It is especially advantageous that such further reduced post-acidification can be obtained even at ambient temperature, such as e.g. at about 20° C. using the mutant strains as described herein.

Unless explicitly indicated otherwise, the various embodiments of the invention described herein can be cross-combined and the described preferences for one of the above aspects of the invention also apply to the other aspects of the invention.

A suitable growth rate of the Lactobacillus bacterial strains according to the present invention and as described herein, such as e.g., Lactobacillus bacterial strain CBS 148322 or preferably the mutant strain CBS 151609, might be measured as the number of colonies of 1 mm diameter or more, preferably of 2 mm diameter or more, even more preferably of 3 mm diameter or more, yet more preferably of 4 mm diameter or more, most preferably of 5 mm diameter or more, i.e., at least about 1, 2, 3, 4 or 5 mm diameter, after 7 days on MRS-agar, wherein the growth rate at 37, 40, 48, 49 or 50° C. being equal to or less than 50%, i.e. at most about 90, 80, 70 or 50%, of the growth rate at 37° C. or at 48° C.

The mean colony diameter of said Lactobacillus bacterial strains measured after 7 days of growth on MRS-agar, with the mean diameter at 37° C., and more preferably at 40° C., might be equal to or less than 90%, more preferably equal to or less than 80%, even more preferably equal to or less than 70%, and most preferably equal to or less than 50%, i.e. at most about 90, 80, 70 or 50%, of the mean colony diameter at 48° C.

The growth rate of the Lactobacillus bacterial strains according to the present invention and as described herein, such as e.g., Lactobacillus bacterial strain CBS 148322 or preferably the mutant strain CBS 151609, might be furthermore measured as the amount of biomass Lactobacillus bacterial strain assessed by optical density or plate counts (CFU/mL) after, in order of preference, 1, 2, 3, 4, 5, 6 or 7 days of growth on MRS-broth, wherein the amount of biomass at 37° C., and more preferably at 40° C., is equal to or less than 90%, more preferably equal to or less than 80%, even more preferably equal to or less than 70%, and most preferably equal to or less than 50%, i.e. at most about 90, 80, 70 or 50%, of the amount of biomass at 46° C., more preferably at 48° C.

With regards to the optimum growth rate of the Lactobacillus bacterial strains according to the present invention and as described herein, such as e.g., Lactobacillus bacterial strain CBS 148322 or preferably the mutant strain CBS 151609, as measured by the amount of biomass of the Lactobacillus bacterial strain in grams after, in order of preference, 1, 2, 3, 4, 5, 6 or 7 days of growth on MRS-agar at a certain temperature, the optimum growth rate might occur at a temperature equal to or above 43° C., even more preferably equal to or above 44° C., yet more preferably equal to or above 45° C., still even more preferably equal to or above 46° C., and most preferably equal to or above 48° C., i.e. an optimal growth rate at least about 43, 44, 45, 46 or 48° C.

In one embodiment the Lactobacillus bacterial strain as described herein, such as e.g., Lactobacillus bacterial strain CBS 148322 or preferably the mutant strain CBS 151609, has an optimum growth rate, as measured by the amount of biomass of the Lactobacillus bacterial strain in grams after, in order of preference, 1, 2, 3, 4, 5, 6 or 7 days of growth on MRS-agar, wherein the optimum growth rate lies outside the temperature range of 37° to 43° C., preferably outside the range of 35° to 45° C.

The Lactobacillus bacterial strain according to the present invention and as described herein, such as e.g., Lactobacillus bacterial strain CBS 148322 or preferably the mutant strain CBS 151609, might have the ability to grow one or more colonies of 1 mm diameter or more at a temperature of 48° C. or higher within 7 days. More preferably, the Lactobacillus bacterial strain might have the ability to grow one or more colonies of 1 mm diameter or more at a temperature of 49° C. or higher within 7 days. Particularly, the Lactobacillus bacterial strain might have the ability to grow one or more colonies of at least 1 mm diameter at a temperature of at least about 50° C. within 7 days.

### FIGURES

FIG. 1: impact of incubation temperature on the number of viable cells of ancestral strain CBS 148322.

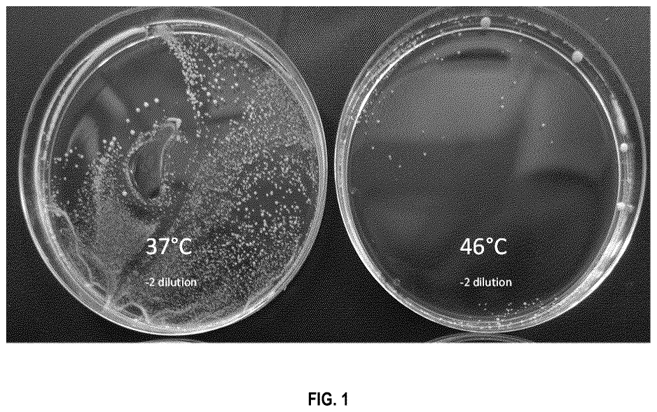

The following examples are illustrative only and are not intended to limit the scope of the invention in any way. The contents of all references, patent applications, patents, and published patent applications, cited throughout this application are hereby incorporated by reference, in particular EP3279312, WO2013/153074, WO2012/136830, PCT/EP2022/080223, and WO2019/043055.

### EXAMPLES

### Example 1: General Methods and Materials

All basic molecular biology and DNA manipulation procedures described herein are generally performed according to Sambrook et al. (eds.), Molecular Cloning: A Laboratory Manual. Cold Spring Harbor Laboratory Press: New York (1989) or Ausubel et al. (eds). Current Protocols in Molecular Biology. Wiley: New York (1998).

Strains. Bioprotective strains used are strain CBS 148322 (as described in PCT/EP2022/080223), strain CBS 149825, and strain CBS 151609, all deposited at Westerdijk Fungal Biodiversity Institute (The Netherlands) under the provisions of the Budapest Treaty.

Contaminants used in the challenge tests are selected from different fungal strains as indicated in Table 3 and as obtainable from public culture collections.

Commercial starter culture for yogurt was selected from YS-141 (DSM Food and Beverages, Delft, the Netherlands).

Adaptive Laboratory Evolution (ALE). For generation of mutant Lactobacillus strains, particularly strain CBS 151609, ALE is performed with strain CBS 148322 as mother strain, with generation of an initial population with or without genotypic diversification (e.g., by mutagenesis), followed by evolution under selected growth conditions, for a desired time, or until the population exhibits a desired phenotypical change.

After several rounds of ALE, in which the population has evolved such that a changed phenotype has become apparent, populations and/or isolates are analyzed for beneficial mutations. The resulting isolates can be used directly as they are or serve as input for (a) next round(s) of ALE.

Milk fermentation. 12% (w/v) RSM (reconstituted skim milk) was pasteurized by heating in a water bath for 20 minutes at 100° C. After pasteurization, the milk was quickly cooled in ice-water and kept at 4° C.

Bioprotective adjunct strains were precultured by inoculating MRS (de Man-Rogosa-Sharpe) broth from frozen stocks of bacteria and incubating them at 37° C. overnight. Prior to inoculation the culture was washed once with physiological saline. The pasteurized milk was inoculated with the bioprotective adjunct cultures at an inoculation rate of 10⁷ CFU/mL. In addition, 1 U/1000 L of a yogurt starter culture Delvo® Fresh YS-141 (comprising Streptococcus thermophilus and Lactobacillus delbrueckii bulgaricus, commercially obtainable from DSM, Delft, the Netherlands) were added. The inoculated milk was incubated at 42° C. The pH was continuously monitored using a CINAC apparatus (Ysebaert, France). Alternatively, acidification was performed in microplates using fluorescent pH probes for measuring the acidification. Several methods have been published and are known to those skilled in the art.

Simultaneously, for the purpose of additional measurements, samples have been prepared in an identical manner. For instance, in cups (e.g., 150 ml volume, diameter 5.5 cm), in tubes (e.g., 50 mL) or in microplates.

Samples for additional analysis (for instance: post-acidification, texture analysis, and/or challenge tests for bioprotective activity) were harvested when the pH of the fermented milk reached pH 4.6. The fermented milks thus obtained were cooled and stored at 4° C. until further analysis (yogurt samples).

Post-acidification (PA) measurement. The degree of post-acidification was determined by measurement of the pH of the yogurt samples in time. To this end, the yogurt samples were incubated at different temperatures to mimic storage of the product, at for instance 7° C. and 20° C. (see Example 3). For each time point, at each temperature, a separate small sample (1-10 mL) is being prepared, which was discarded after the pH measurement.

Challenge test. The fermented milk products were subjected to a challenge test, to show the bioprotective activity of the bioprotective adjunct CBS 151609. Each sample was divided: one part was not contaminated; one part was contaminated with mould spores of strains as indicated in Table 3. The contaminants were added to the fermented milk products at about 50 spores per spot. Both species are well-known contaminants in the dairy industry.

The fungal spores were pipetted on top of the yogurt samples that were previously mixed with an equal volume of agar melted in water, and that were allowed to cool and solidify in plates (about 50 g per plate).

The cups, tubes or microplates were closed with appropriate lids and stored at the desired temperature (for instance, 20° C.; see Example 3). Cups were inspected and photographed at regular intervals (days, weeks) to look for the occurrence of mould growth on the surface of the products, up to 12 days.

### Example 2: Growth Performance and pH of Mutant Strains Derived from ALE

Several mutants of Lactobacillus casei strain CBS 148322 were generated via ALE and tested in a CINAC-scale analysis (see Ex. 1) to check feature points of the milk fermentation, i.e., time to reach pH 4.6 (TTR pH 4.6) and pH value at 20 hours timepoint.

Yogurts were made using 1 U/1000 L of a starter culture (Delvo® Fresh YS-141, commercially available from DSM, Delft, The Netherlands) comprising Streptococcus thermophilus and Lactobacillus delbrueckii bulgaricus, supplemented with or without a bioprotective culture (bioprotective adjunct), i.e. strain CBS 148322 as mother strain (ancestor) or mutants derived from ALE, i.e. mutant #1, #2, #3, #4, whereby mutant #3 was later deposited as strain CBS 151609. Milk fermentation was performed as described in Ex. 1, with inoculation of overnight cultures of the bioprotective cultures at an inoculation rate of 10⁷ cfu/ml. As soon as the pH reached the value of 4.6, the fermentation was stopped by cooling the fermented milk for 15 min in ice water. The results are shown in Table 1.

TABLE 1
Acidification (“TTR pH 4.6”) of yogurts comprising starter
culture YS-141 and a bioprotective LAB strain (“BP adjunct”)
at 42° C. “None” means the starter culture only without any
BP-adjunct, “ancestor” means strain CBS 148322 as BP-
adjunct, “mutant” means mutant strain derived from ALE added
as BP-adjunct. The results are given as average ± standard
deviation of 3 biological replicates. For more details, see text.

TTR pH 4.6

| BP adjunct | minutes | hours |
|---|---|---|
| ancestor | 377 ± 39 | 6.3 ± 0.6 |
| mutant #1 | 350 ± 7 | 5.8 ± 0.1 |
| mutant #2 | 355 ± 3 | 5.9 ± 0.05 |
| mutant #3 | 375 ± 22 | 6.3 ± 0.4 |
| mutant #4 | 398 ± 30 | 6.6 ± 0.5 |

As shown in Table 1, the speed of yogurt fermentation as indicated by TTR pH 4.6 is similar when the mutants and the ancestor strain CBS 148322 are added as BP-adjuncts.

### Example 3: Long-Term PA-Performance of Mutant Strains

Shelf-life of mutant strains (mutant #1, 2, 3, 4) were tested at 20° C. (see Table 2A) as well as 7° C. (see Table 2B) in a yogurt with probes taken at the start, i.e., day 0, and after 1, 4, 6, 8, 11 or 13 days, respectively. The experimental set-up was as described above. The results are shown in Table 2, with the standard deviation in all cases being in the range of 0.01 or less.

TABLE 2A
PA at 20° C. using the ancestor strain CBS 148322 or
different mutant strains as BP adjunct. A yoghurt comprising
starter culture YS-141 only (without any BP adjunct) is
taken as reference (“none”). The results are given
as average of 3 biological replicates. For more details, see text.

Days

| BP adjunct | 0 | 1 | 4 | 6 | 8 | 11 | 13 |
|---|---|---|---|---|---|---|---|
| none | 4.6 | 4.6 | 4.4 | 4.3 | 4.3 | 4.2 | 4.2 |
| ancestor | 4.6 | 4.5 | 4.1 | 3.9 | 3.9 | 3.8 | 3.8 |
| mutant #1 | 4.6 | 4.5 | 4.2 | 4 | 3.9 | 3.9 | 3.8 |
| mutant #2 | 4.6 | 4.5 | 4.1 | 4.0 | 3.9 | 3.8 | 3.8 |
| mutant #3 | 4.6 | 4.6 | 4.3 | 4.2 | 4.1 | 4.0 | 4.0 |
| mutant #4 | 4.6 | 4.5 | 4.2 | 4.0 | 3.9 | 3.9 | 3.8 |

At 20° C., the addition of bioprotective mutant #3 performed better than the addition of its ancestral strain, the known bioprotective strain CBS 148322. When compared to a yogurt comprising the starter culture only and no BP-adjunct, addition of mutant #3 (i.e., strain CBS 151609) resulted in a pH decrease of only 0.2 after 13 days at 20° C. as compared to a BP-free yogurt (see “none” in Tab. 2A). Under the same conditions, addition of the ancestor strain CBS 148322 or mutants #1, #2 or #4 into a yogurt leads to a pH reduction of 0.4 compared to the pH in a yogurt comprising the starter culture only and no bioprotective adjunct (see “none” in Tab. 2A).

Under the same conditions, strain CBS 148322 was tested against a Lactobacillus strain CBS 149825 and the pH measured at day 1 (pH 4.3 vs 4.4), day 3 (pH 4.1 for both), day 7 (pH 3.8 for both), and day 14 (pH 3.7 vs 3.8), thus a reduction of post-acidification by 0.1 for strain CBS 149825 after 14 days at 20° C. compared to strain CBS 148322. A sample without any bioprotective adjunct (referred to as “none” in Table 2A above) showed a pH of 4.1 after 14 days at 20° C. (data not shown).

TABLE 2B
PA at 7° C. using the ancestor strain CBS 148322 or
different mutant strains as BP adjunct. A yoghurt comprising
starter culture YS-141 only (without any BP adjunct) is
taken as reference (“none”). The results are given
as average of 3 biological replicates. For more details, see text.

| | Days | | | | | | | |
|---|---|---|---|---|---|---|---|---|
| BP adjunct | 0 | 1 | 4 | 6 | 8 | 11 | 14 | 21 |
| none | 4.6 | 4.7 | 4.6 | 4.5 | 4.5 | 4.5 | 4.4 | 4.4 |
| ancestor | 4.6 | 4.5 | 4.4 | 4.4 | 4.3 | 4.3 | 4.2 | 4.2 |
| mutant #1 | 4.6 | 4.5 | 4.5 | 4.4 | 4.4 | 4.3 | 4.3 | 4.2 |
| mutant #2 | 4.6 | 4.5 | 4.5 | 4.4 | 4.4 | 4.3 | 4.3 | 4.2 |
| mutant #3 | 4.6 | 4.6 | 4.5 | 4.4 | 4.4 | 4.4 | 4.3 | 4.3 |
| mutant #4 | 4.6 | 4.5 | 4.5 | 4.4 | 4.4 | 4.3 | 4.3 | 4.2 |

Comparing the shelf-life of yogurt at 7° C., the addition of bioprotective mutant #3 (i.e., strain CBS 151609) performed better than the addition of its ancestral strain, CBS 148322. Compared to a yogurt comprising the starter culture only and no BP-adjunct (see “none” in Tab. 2B), addition of mutant #3 (i.e., strain CBS 151609) resulted in a pH decrease of only 0.1 after 21 days. Under the same conditions, addition of the ancestor strain CBS 148322 or mutants #1, #2 or #4 into a yogurt leads to a pH reduction of 0.2 compared to the pH in a yogurt comprising the starter culture only and no bioprotective adjunct (see “none” in Tab. 2B).

### Example 4: Inhibition of Fungal Growth by Mutant Strains

A challenge test was performed to test the bioprotective activity of the mutant strains independently of the decreased contribution to the PA (see Ex. 1). The results are shown in Table 3.

TABLE 3
bioprotection against selected mould and yeast strains as indicated (“Fungal
contaminant”) and measured after incubation of a yogurt sample at 20°
C. for 11 days, wherein the performance of the ancestor strain CBS 148322
(“ancestor”) is compared to mutant strains #1, 2, 3 or 4 (mutant
#1 has been deposited as strain CBS 151609) added as bioprotective
adjunct to a yogurt comprising starting culture YS-141.

| Fungal contaminant | | BP adjunct | | | | |
|---|---|---|---|---|---|---|
| type | Type strain | ancestor | #1 | #2 | #3 | #4 |
| mould | Aspergillus niger ATCC 16404-1V | +++ | +++ | +++ | +++ | +++ |
| | Fusarium oxysporum VTT D-80134 | +++ | +++ | +++ | +++ | +++ |
| | Penicillium brevicompactum CBS 110070 | ++ | +++ | +++ | ++ | +++ |
| | Penicillium italicum ATCC 36041A | ++ | ++ | ++ | ++ | ++ |
| yeast | Toluraspora delbrueckii CBS 1148 | +++ | +++ | +++ | +++ | +++ |
| | Debaryomyces hansenii ATCC 18110A | +++ | +++ | +++ | +++ | +++ |
| | Pichia ciferrii ATCC 32292A | +++ | +++ | +++ | +++ | +++ |
| | Torulaspora delbrueckii CTS-GCY-A | + | ++ | ++ | + | ++ |
| | Zygosaccharomyces bailii CBS 1097AA | − | +++ | − | − | − |
| | Saccharomyces cerevisiae CEN.PK113-1A | ++ | +++ | +++ | ++ | +++ |

“−” means no inhibition, “+” means weak inhibition, “++” means strong inhibition and “+++” means complete inhibition of growth of contaminants as indicated in the table. For more details, see text.

The bioprotective activity of the mutant strains #1, 2, 3 and 4 was more or less in line with the ancestral strain CBS 148322, in addition to the fact that particularly mutant strain #3 (i.e., strain CBS 151609) has better post-acidification properties.

1. A method for obtaining a mutant lactic acid bacterial strain, optionally a Lactobacillus bacterial strain, optionally capable of reducing post-acidification in a fermented milk product in comparison with a parent Lactobacillus bacterial strain, wherein the method comprises subjecting the parent lactic acid bacterial strain to an adaptive evolution, said adaptive evolution comprising a series of evolutionary changes, wherein each next descendent strain of a preceding strain is subjected to a higher environmental temperature than the preceding strain and wherein the mutant lactic acid bacterial strain when present in or added to a fermented milk product is increasing the pH in said fermented milk product in the range of 0.1 to 0.2 during storage of the fermented milk product at about 20° C. as compared to the pH measured under the same conditions in a fermented milk product comprising the parent Lactobacillus bacterial strain, and wherein the pH is determined after storage of said fermented milk product fermented with a starter culture over at least about 14 days at 20° C., and wherein said parent or mutant Lactobacillus bacterial strain is present in a concentration of at least about 10⁷ cell forming units per gram (cfu/g).

2. The method according to claim 1, wherein the parent and the mutant Lactobacillus bacterial strain is selected from the group consisting of a Lactobacillus rhamnosus strain, a Lactobacillus casei strain, a Lactobacillus plantarum, and a Lactobacillus paracasei strain.

3. The method according to claim 1, wherein the parent and the mutant Lactobacillus bacterial strain has antimicrobial, antifungal and/or anti-yeast properties.

4. The method according to claim 1 for production of a Lactobacillus bacterial strain with antimicrobial, antifungal and/or anti-yeast properties.

5. The method according to claim 1, wherein the parent strain is strain CBS 148322 and the mutant strain is selected from strain CBS 151609.

6. A mutant Lactobacillus bacterial strain obtainable by the method according to claim 1.

7. The mutant Lactobacillus bacterial strain according to claim 6, which is strain CBS 151609.

8. The mutant Lactobacillus bacterial strain according to claim 6, said strain having the same or improved antimicrobial, antifungal and/or anti yeast properties as strain CBS 148322.

9. The mutant Lactobacillus bacterial strain according to claim 6 being present in frozen, dried, or freeze-dried form.

10. An antimicrobial and/or antifungal and/or anti-yeast composition comprising the mutant Lactobacillus bacterial strain according claim 6.

11. A food product comprising the mutant Lactobacillus bacterial strain according claim 6 or an antimicrobial and/or antifungal and/or anti-yeast composition thereof.

12. The food product according to claim 11, wherein the food product is a fermented milk product, optionally cheese, sour cream, or yogurt.

13. A process for manufacturing the food product according to claim 11, comprising adding the mutant Lactobacillus bacterial strain, or an antimicrobial and/or antifungal and/or anti yeast composition thereof, during manufacture of the food product.

14. A method of providing an antimicrobial and/or antifungal and/or anti yeast effect in a food product, optionally in a fermented milk product, the method comprising inoculating milk with the mutant Lactobacillus bacterial strain according to claim 6.
